(19) United States
(12) Patent Application Publication
(10) Pub. No.: US 2004/0175843 A1
(43) Pub. Date: Sep. 9, 2004

(54) Near-field and far-field encoding and shaping of microbeads for bioassays

(76) Inventors: Daniel B. Roitman, Menlo Park, CA (US); Annette Grot, Cupertino, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/669,620

(22) Filed: Sep. 24, 2003

Related U.S. Application Data
(63) Continuation-in-part of application No. 10/379,107, filed on Mar. 4, 2003.

(51) Int. Cl. G01N 33/545

(52) U.S. Cl. 436/531

(57) ABSTRACT

An encoded patterned microbead of polymeric material, with an associated geometry, capable of linking to a ligand molecule, processes for fabricating shaped and patterned microbeads, a reader to read the patterned microbead, and methods to produce and read the shaped and patterned microbead are disclosed. A unique identifier is written to the encoded patterned microbead and the encoded patterned microbead is given an identifying shape according to one of several well-known techniques. A reader of the present invention, as well as conventional readers, read the shaped, encoded, patterned microbeads.

### CROSS REFERENCE TO RELATED APPLICATIONS

The present application is a continuation-in-part of U.S. patent application Ser. No. 10/379,107, filed Mar. 4, 2003, entitled NEAR-FIELD AND FAR-FIELD ENCODING OF MICROBEADS FOR BIOASSAYS, incorporated herein in its entirety by reference.

### BACKGROUND OF THE INVENTION

This invention relates generally to combinatorial chemistry and analyte binding, and more specifically to a microbead that is encoded and shaped to enable a high degree of multiplexing, a method for making such a microbead, and a reader to read the microbead.

The use of microbeads for combinatorial chemistry and multiplexed sensors is based on four conditions. One condition is that the microbead surface can be suitably modified for molecular recognition. Another condition is that there is an encoding method that identifies uniquely the class corresponding to a particular microbead. A third condition is that there is an effective method to read the encoded information. The final condition is that there is an effective method to read with sensitivity and some degree of quantification the analyte binding. Clearly these conditions can be interrelated. The surface that is suitable for molecular recognition, for example, may also be suitable for encoding a unique identifier. The method for reading the encoded information can be dependent upon the method of encoding and on the shape of the microbead.

Several technologies exist that provide parallel assay multiplexing. One of these techniques, known as spatial multiplexing, involves the use of a microarray in which the location of each individual “assay” (corresponding to each spot) in the array provides the required unique encoding. In this technology, analyte molecules such as nucleic acids and proteins can be detected, identified, and quantified when thousands of different ligand molecules that bind specifically to analytes are immobilized as spots in a defined pattern on the surface of a substrate. When a sample is introduced and the ligand-analyte pairing (such as the complementary strands of nucleic acids) occurs at specific locations within the microarray, the identity of the hybridized (or annealed) part of the sample, the part that contains the analyte molecule, can be deduced from the location of the corresponding hybridization “spot” within the microarray. However, this technique presents several drawbacks. One is that the reproducibility from spot to spot and from array to array is difficult to assess, since each spot is individually created by some form of printing technique. Another drawback is that the substrate is common to all the spots of the array, and there is no chemical flexibility or chemical freedom to select different chemistries for different ligands or analytes. Another drawback is the difficulty to automate and integrate the assays with existing sample handling techniques (such as microtiter plates and microfluidics systems), mass spectrometry, and downstream sample analysis. Another drawback is that mixing of reagents and analytes is not as effective in planar configurations as in wells or test tubes.

In order to decouple each assay (or spot) of an array from positional identification, another technique can be used in which each individual assay itself is carried out on a labeled microbead. Labeling can be accomplished by tagging the microbead with dyes, a process known as color or spectral multiplexing. Roughly spherical microbeads, typically plastic-based, are encoded by the incorporation of photo luminescent materials. Encoding is achieved by spectral characterization and intensity multiplexing. The microbead surfaces are typically modified with conjugating groups capable of immobilizing ligands for analyte capture. The capture of the analytes is typically revealed by dye-conjugation of the analytes or by sandwich assays with a secondary fluorescent ligand, nanoparticles, or enzymes capable of some form of signal transduction. The reading of the code and the binding events are typically accomplished by spectrally resolved photo luminescence.

An advantage of photo luminescent encoding is its capability of relatively easy detection achieved using conventional “flow cytometry” instrumentation. Also, photo luminescent encoding enables the use of a wide range of microbead sizes, for example from 1-100 μm. Plastic microbeads have relatively low densities (around 1.3 g/cc) and are relatively easy to formulate as dispersions and colloidal suspensions. However, the number of distinct codes achievable with color and intensity multiplexing is currently limited to about 100 because there are typically two colors involved, and ten intensity levels within each color. Adding a third color is possible, but challenging for numerous technical reasons (it requires the use of multiple lasers, careful characterization and minimization of artifacts such as “cross-talk” between dyes, non-uniform dye distribution). It is difficult to multiplex spherical microbeads because the emission bandwidth and quantum efficiency of the dyes limit the choices to two or at most three colors, and the intensity levels are difficult to fine-tune to ten or more distinct levels. In addition the dyes should not overlap with the biomolecular tag or transfer energy with it nor among themselves. The biomolecular tag is preferentially “blue”-shifted relative to the bead-encoding dyes, and this limits the choices of suitable biomolecular dyes. Finally, multiple lasers are typically required since each dye has a characteristic and different excitation spectrum.

Another process for identifying an analyte molecule involves semiconductor nanocrystals, or quantum dots. Quantum dots can be incorporated into polymeric microbeads at precisely controlled ratios. Each dot has a characteristic spectral emission that can be tuned to a desired energy by varying the particle size, size distribution, and/or composition of the particle. The characteristic emission spectrum can be observed spectroscopically. A drawback with this technique is that is challenging to incorporate quantum dots into plastic microbeads in a reproducible manner. Although quantum dots do not require multiple lasers and they have narrower emission spectra than dyes, they are difficult to manufacture with reproducible optical properties (both in color and quantum efficiency) and to formulate into solvent-compatible suspensions for embedding into plastic microbeads. Also, they are not generally available in the marketplace, and they are expensive. It would be more desirable to encode microbeads with low-cost methods and with existing materials in the marketplace.

Yet another process for identifying an analyte molecule involves rod-shaped particles fabricated by metal deposition inside the pores of a nanoporous membrane followed by the dissolution of the membrane and freeing the rods to provide a large pool of uniquely identifiable encoders. The encoding of rods can be very effectively achieved by alternating metal compositions along the length of the rod, but the readout of the encoded information is difficult because, in part, of the small size of the rods. Fabricated rods from gold and silver are extremely dense, somewhat cumbersome to manufacture in reproducible ways, and will not disperse easily or remain suspended for extended times unless they are very small in diameter, i.e. 300 nm, and length, i.e. 6 to 10 μm. The encoding of rods is read by the reflectivity pattern (barcode) and the analyte is read by dye fluorescence. Larger metals rods are undesirable since their densities are too high to formulate them into stable dispersions. Metal barcodes are relatively difficult to make in a reproducible manner (a template is required for growing the metal rods), the metal surfaces need to be stabilized against corrosion degradation (a problem with silver). Because of the high density of gold and silver (ρ=19.3 and ρ=10.5 g/cm³ respectively) it is challenging to work with them in fluidic systems as their sedimentation rates in water-based buffers (ρ=1 g/cm³) are much faster than for polymeric materials (ρ˜1.1 to 1.5 g/cm³). As a result the metal rods must have features of the order of just 1 μm or less which require special optics to read. If the readout is done with a flow-device, the optical train (slits) needs to resolve micron-sized features at a high speed. If the readout is done on a substrate, specialized powerful high magnification optics capable of resolving 1 μm or less and imaging software is required. Current commercial array scanners are not suitable since their pixel sizes are 5 μm on the side or larger and are designed for fluorescence detection only.

Other prior art describe encoding microlabels fabricated from anodisable material (e.g. aluminum) using microlithography. Prior art microlabels are encoded in one dimension, and thus require a system that understands the alignment of the bars to prepare a proper readout of the information. Furthermore since they do not have cylindrical symmetry, the readout in flow using a slit suffers from further complications as the microbar rotates along its long axis. The material of prior art microlabels is limited to anodized aluminium, and this limits flexibility in manufacturing.

Another approach involves radio frequency transponders that can be powered by light. A laser powers the transponder and excites a tag that is fabricated into the microbead. The tag responds with unique identification of the ligand. Typical tags can return a 64-bit identifier, or 10¹⁹ unique identifiers. These identifiers can be read at a rate of 200 kbit/second, and the tags themselves can be processed by a cytometer-based reader at a rate of about 1000 microbeads/second. These transponders are very effective in multiplexing the information for individual microbead recognition, but they are bulky, e.g. 250 μm, expensive to manufacture, and are of high density (i.e. 5 g/cc) and are thus difficult to disperse.

Microbeads that are encoded in multiple dimensions present an virtually unlimited number of identifiers without substantially increasing system processing time. Encoded microbeads that are etched or lithographically divided and separated into a plurality of microbeads can be read in a number of ways, including by means of a specialized reader. The promise of these microbeads could be fulfilled by increasing the speed and accuracy at which they are read.

### SUMMARY OF THE INVENTION

The problems set forth above as well as further and other problems are resolved by the present invention. The solutions and advantages of the present invention are achieved by the illustrative embodiment described herein below. The present invention in built on the technology described in U.S. patent application Ser. No. 10/072,837, entitled METHODS FOR MAKING MICROBAR ENCODERS FOR BIOPROBES, incorporated herein in its entirety by reference, and

The present invention includes an encoded and shaped microbead or label that is made from micropatterned polymeric material in the form of a polymeric substrate which is etched or lithographically, shaped, divided, and separated into a plurality of microbeads from the polymeric substrate. Additionally, the present invention includes methods to encode the polymeric substrate, a method to create a specialized receiving substrate, and a method to read the shaped microbead. Encoding of the microbead involves varying possible characteristics of the entire microbead, such as, for example, topography, reflectivity, and fluorescence emission, and others, where the encoding is not restricted to a particular dimension of the microbead. Shaping the microbead and the receiving substrate involves several possible techniques described herein for achieving desired possible shapes. The encoded and shaped microbead is suitable for chemical conjugation with ligands.

The microbead material may be micropatterned and shaped by replication using a patterned master substrate made from a suitable rigid material such as silicon, quartz, glass, metals such as stainless steel, copper, nickel, brass, etc. Replication can be achieved by processes such as (1) hot embossing, (2) casting or injection molding the polymeric material in the form of a liquid resin onto the patterned master substrate followed by a hardening step and a release step to free the polymeric substrate now micropatterned, or (3) by forcing the liquid resin by capillary action into a narrow gap defined by the space between the patterned master substrate and another rigid substrate, or between two patterned master substrates, hardening the resin and releasing the polymeric substrate now micropatterned. Replication is not limited to these techniques.

The polymeric material in the form of a single or multilayer polymeric substrate may be micropatterned according to techniques such as those used for storing binary data on removable computer media such as Compact Discs (CDs) or Digital Versatile Disks (DVDs), or the manufacture of an optical grating patterned on or in the polymeric material to create specific reflective or diffractive patterns. The polymeric substrate may also be micropatterned by either photolithographic processes using photosensitive materials such as positive or negative resists, or by a laser using ablation, phase transition, reflection changes, etc. The microbeads may also include a transducing layer that may be polymeric, metallic, or dielectric inorganic material. The microbeads may contain a bleachable substance that, when exposed to light, produces a desired pattern, or the code itself can be encoded through bleaching of the microbead.

The microbeads of the present invention are illustratively constructed in shapes that are significant during the identification process. These shapes are etched or lithographically divided and then separated into a plurality of microbeads from an initially continuous sheet or film of polymeric substrate. The sheet could be either free-standing or coated on top of another substrate. The encoding of the microbeads is carried out before, during, or after the microbeads have been “defined” on the sheet, but always before separating the individual microbeads, i.e. it is done on a continuous area, and handled in batch mode or as a sheet of flexible film (roll to roll processing), then the microbeads are separated from each other and freed from supporting substrates.

The present invention also includes a receiving substrate that has openings that are of predetermined shape designed to receive shaped microbeads of the present invention. Although the substrate can be directly etched to form the desired shapes (e.g. etching a glass substrate with acid), most methods to create the substrate involve at least one layer of material etched on the substrate, and then removed or dissolved leaving the shaped opening behind.

After the microbeads are shaped and encoded, one possible method to read them involves suspending them in a fluid and flowing the fluid and microbeads over the receiving substrate. The microbeads are deposited into the receptor regions using fluidic deposition such that each shaped microbead is suitably matched and oriented within a receptor region. Microbead reading can be accomplished by a conventional near-field optical system such as a fluorescent microscope or more sophisticated near-field readers. Another alternative for reading the microbeads involves projecting a beam of light onto one or several microbeads that have been patterned with optical gratings. The reflected or diffracted light emerging from the microbeads is projected onto a surface, and the microbead's information is read from that surface. A far-field sensor can thus be used to gather analyte information.

For a better understanding of the present invention, together with other and further objects thereof, reference is made to the accompanying drawings and detailed description. The scope of the present invention is pointed out in the appended claims.

### DESCRIPTION OF THE SEVERAL VIEWS OF THE DRAWING

FIGS. 1A-D illustrate, schematically, the receiving substrate of the present invention;

FIGS. 2A-2F illustrate, schematically, the microbead formulation of the illustrative embodiment of the present invention in which the polymeric material is cast onto a patterned master;

FIGS. 2G-2L illustrate, schematically, the microbead formulation of an alternate embodiment of the present invention in which the polymeric material is embossed;

FIGS. 2M-2S illustrate, schematically, the microbead formulation of a second alternate embodiment of the present invention in which soft polymeric material is imprinted with a two-level pattern;

FIGS. 2T-2W illustrate, schematically, the microbead formulation of a third alternate embodiment of the present invention in which laser ablation in used to inscribe polymeric material;

FIGS. 2X-2Z illustrate, schematically, the microbead formulation of a fourth alternate embodiment of the present invention in which the microbead hosts a digital data layer that is physically pitted in a desired pattern;

FIG. 3A schematically, pictorially illustrates a rotationally invariant diffractive optics pattern encoded on a microbead created to be read by a reader of the illustrative embodiment of the present invention;

FIG. 3B is a microphotograph of a diffractive optics pattern that, when illuminated, generates a 4×4 array of light, encoded on a microbead created to be read by a reader of the illustrative embodiment of the present invention;

FIG. 3C schematically illustrates a DVD or CD pattern encoded on a microbead created according to the method of the illustrative embodiment of the present invention;

FIGS. 4A-4C schematically illustrate another microbead formulation that is within the illustrative embodiment of the present invention;

FIGS. 5A-5C are schematic, pictorial representations that illustrate a master holding resulting microbeads, a microbead after lift-off, and a layered microbead after lift-off respectively, after encoding by the illustrative embodiment of the present invention; and

FIG. 6 illustrates a schematic, pictorial system for reading microbeads that are encoded by optical grating techniques.

### DETAILED DESCRIPTION OF THE INVENTION

The present invention is now described more fully hereinafter with reference to the accompanying drawings, in which the illustrative embodiment of the present invention is shown.

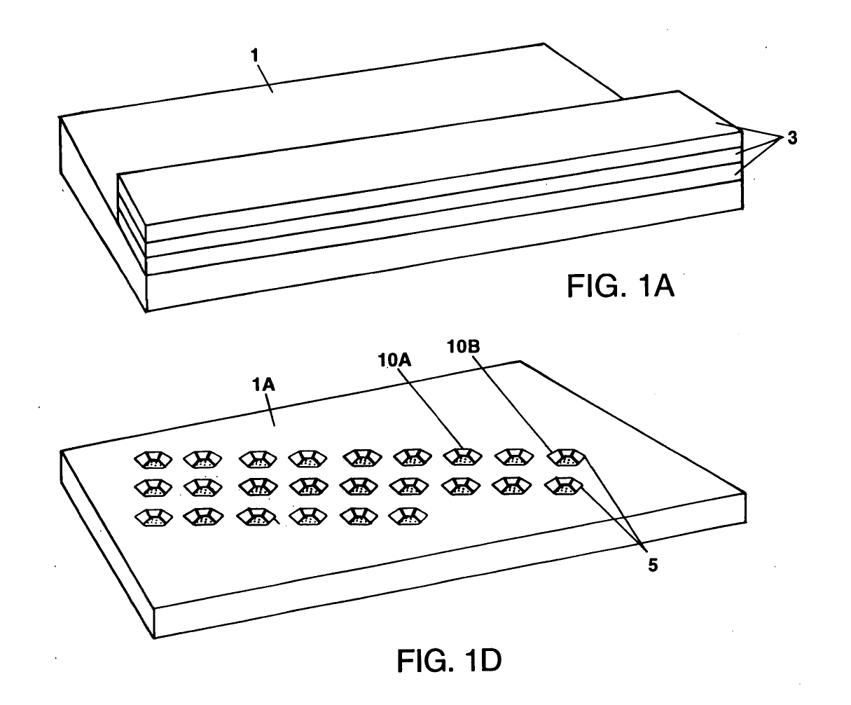

FIGS. 1A-1D illustrate the creation of a receiving substrate into which encoded shaped microbeads can be deposited during fluidic deposition. In FIG. 1A, a substrate 1 can be obtained on which to form a set of recessed receptor regions which are wells or indentations that match or complement the shape and thickness of the microbeads. The receiving substrate 1, containing any number of layers 3, may be formed of material such as, for example, glass, plastic, multiple plastics such as, for example, PMMA, any material that can be thermally cured or photo cured, moldable materials such as, for example, thermoplastics or polymeric material, transparent or opaque, hard materials, silicon, inorganic materials, or metals, such as, for example, nickel. The receiving substrate 1 can be formed by techniques such as, for example, embossing, photolithography, etching, injection molding, or any other suitable method. Any layers 3 may be formed by techniques such as, for example, spray coating, spin casting, dipping, sputtering, plasma enhanced chemical vapor deposition (PECVD), sol-gel chemistry, or any other suitable method.

Referring now to FIG. 1B, receiving substrate 1 (FIG. 1A) can be fabricated with wells or indentations, receptor regions 5, into which encoded, shaped microbeads can be deposited during fluidic deposition. Receptor regions 5 may be formed using a technique such as, for example, template punch, laser, chemical or plasma etching, casting, or impact extrusion. Particular shapes can be achieved by placing a patterned mask upon a single layer of material having special characteristics such that exposing the mask at an oblique angle can also expose the material underlying the mask, forming a specific type of opening in the substrate, such as, for example, a trapezoidal with smaller side 7 and larger side 6. Receptor regions 5 can be formed so that the encoded, shaped microbeads can be deposited into receptor regions 5 in an orientation such that it could be possible to read the encoding of the microbeads.

Although it could be possible to shape receptor regions 5 in any shape at all, the lower the number of possible orientations that microbeads can fall into receptor regions 5, the faster the microbeads might be read. Thus, when deciding the shape of the microbeads and receptor regions 5, at one end of the spectrum could be a spherical-shaped (totally symmetric) receptor region 5 and microbead, while at the other end of the spectrum could be an asymmetrically-shaped receptor region 5. Microbeads can more easily fall into symmetrically-shaped receptor regions 5, but asymmetrically-shaped receptor regions 5 can possibly allow for more flexibility in manufacture and encoding. Note that it may not be necessary for the pattern encoded on the microbeads to match the symmetry of receptor regions 5.

Optionally, and referring again to FIG. 1B, receptor region 5 could have, for example, chemical, optical, electrical, or ligand receptor treatment to either attract the microbeads or that matches receptors on the surface of the microbeads (in the case of ligand receptors). Further, an electrostatic or magnetic field in the receptor region 5 could be used to attract the microbeads, although this attraction may not be necessary for the process to reach a successful completion. If a ligand receptor pairing is desired, receptor region 5 could be deepened to minimize the chance of an accidental incorrect ligand-receptor pair. Note that receptor region 5 may not be required to be a physical indentation. For example, receptor region 5 could be formed of a surface treatment such as hydrophilic/hydrophobic, ligand, or electrostatic in a particular shape. Also receptor region 5 could be formed of protrusions which could be mated with holed or divotted microbeads. The invention is not limited to these examples of non-indented receptor regions.

Referring now to FIG. 1C, particular shapes can also be achieved by etching two, possibly different, patterned mask layers at two different times, or patterning three layers, two of which can have a similar ingredient such as silicon dioxide, and the third can be mainly composed of a different ingredient such as metal. The method can involve patterning the top layer (e.g. the metal layer) and middle layer (e.g. a silicon dioxide layer), thus exposing the third layer for etching. Particular shapes can further be achieved by ablating the top layer of a two-layer substrate, or by layering a single layer on a suitable substrate and forming an opening in the layer. As shown in FIG. 1C, the etching can be accomplished in two steps involving two or more layers. Shallow etching 8 can result from, for example, the first of the steps, while deeper etching 9 can result from, for example, the second of two steps. A properly-shaped microbead falling into the recess formed by shallow etching 8 and deep etching 9 may not rest upside-down or otherwise disoriented.

As illustrated in FIG. 1D, the receptor regions 5 may be shaped, spaced, and arranged in any way, so as to accommodate a variety of shapes and a variety of desired recessed receiving substrate 1A configurations. In operation, a slurry containing encoded, shaped microbeads can be flowed over recessed receiving substrate 1A, and the microbeads fall into receptor regions 5 properly oriented. Receptor regions 5 could take shapes such as, for example, but not limited to, trapezoidal 10A, hexagonal 10B, keyed, or notched. In such cases where three-dimensional orientation is a requirement, receptor regions 5 (and encoded, shaped microbeads) could, for example, be irregularly-shaped, with some or all sides having differing lengths. Further, receptor regions 5 and encoded, shaped microbeads could be keyed to achieve certain other effects.

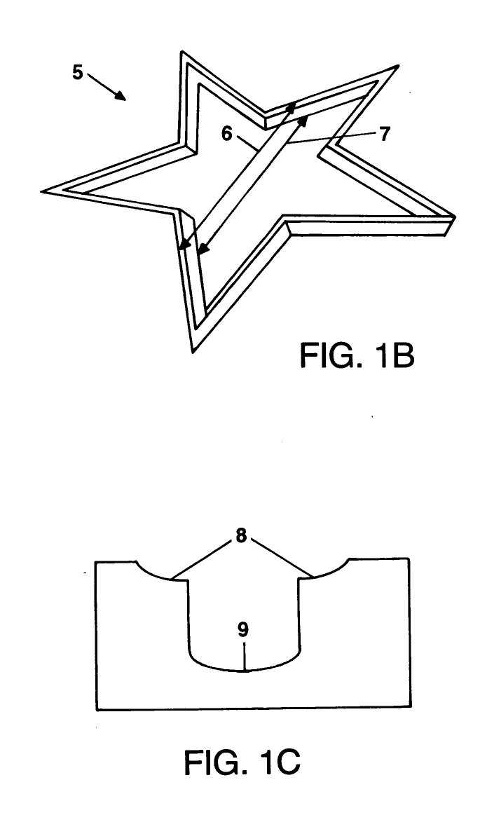

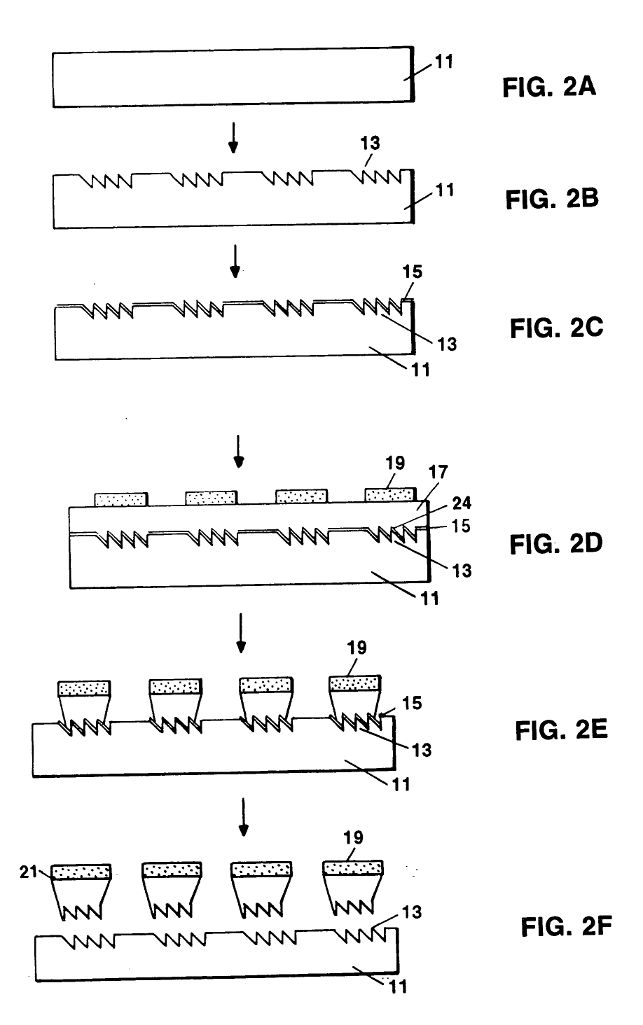

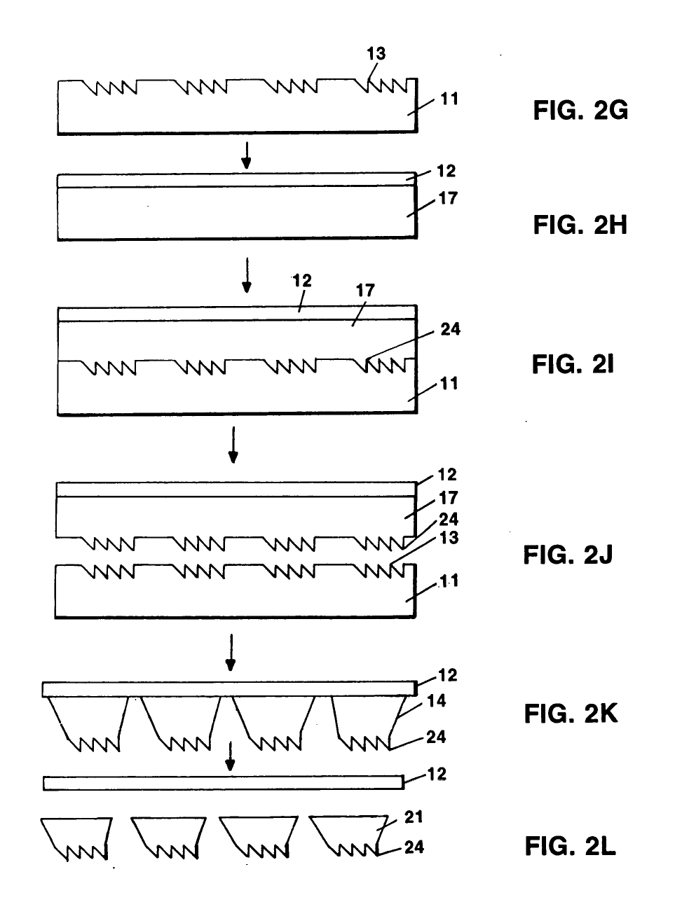

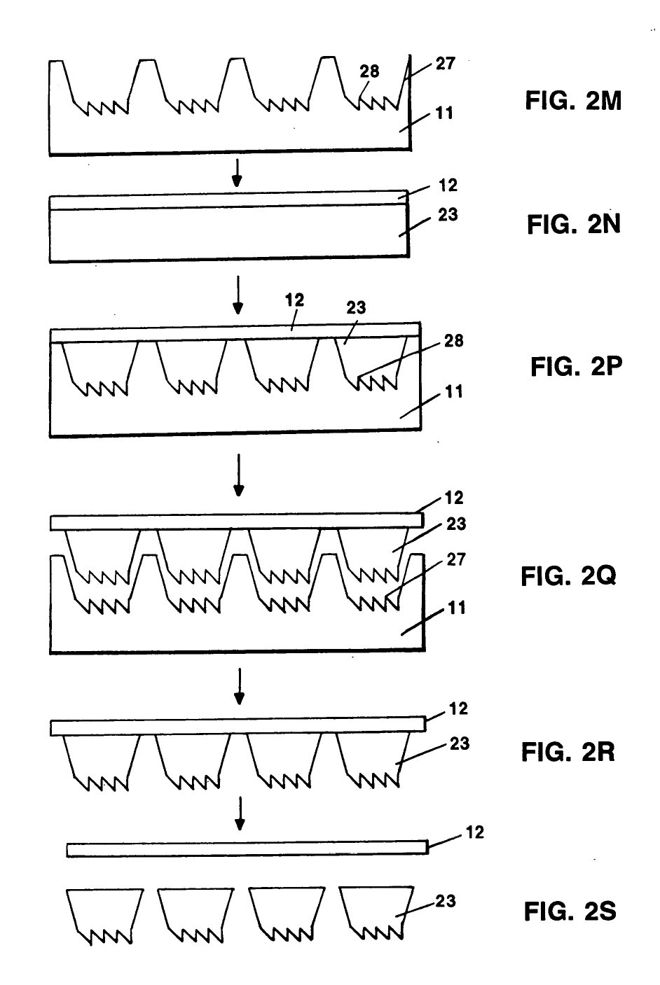

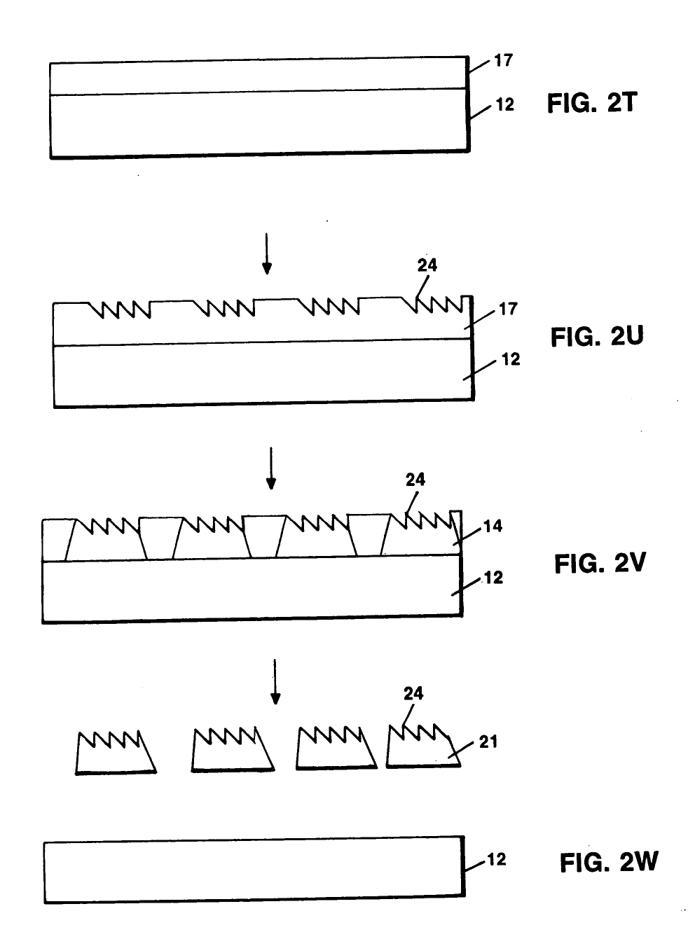

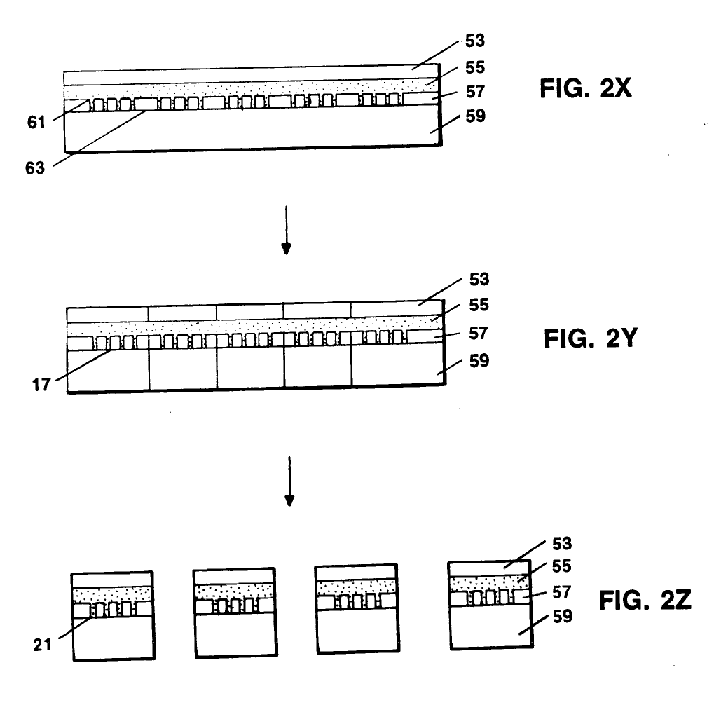

FIGS. 2A-2F illustrate the encoded patterned microbead of the illustrative embodiment of the present invention. In particular, referring to FIG. 2A, beginning with a master substrate 11, in FIG. 2B a relief pattern 13 can be inscribed into master substrate 11 by conventional means. Referring to FIG. 2C, layered on top of master substrate 11 can be a release/sacrificial layer 15, the purpose of which is to allow easy removal of the microbeads after etching. Referring now to FIG. 2D, microbead material 17 such as, for example, polymeric material, can be deposited or injection molded on top of release/sacrificial layer 15 in a, preferably, moldable state (either as a solution, a melt, a cross-linkable resin, or a thermoplastic polymer above the temperature of glass transition (T_{g}) or melting temperature (Tₘ)), and can then be hardened afterwards by a conventional procedure such as solvent evaporation, thermal curing, photo-curing, or by lowering the temperature below T_{g} or Tₘ. Polymeric material 17 can be molded thereupon in the microbead encoding pattern 24. The result can be a replica of the relief pattern 13 written on polymeric material 17. Mask layer 19, deposited in a pattern that can be used to etch or photolithographically define the individual microbeads, can be laid on top of polymeric material 17. Mask layer 19 can be any shape and size. During this step, conventional microlithography alignment techniques (for example, but not limited to, those described in published U.S. Patent Application 2002/0098426 incorporated herein in its entirety by reference) can be used to insure that the microbeads are etched or photolithographically defined directly above the inscribed relief pattern 13 of polymeric material 17 so that the proper information can be inscribed upon each microbead. Referring now to FIG. 2E, patterned polymeric material 17 can then be etched appropriately, such as, for example, trapezoidally, to insure that the microbeads fall into the previously-described recesses in the proper orientation, such that their encoding can be read. Patterned polymeric material 17 can then be lifted from a support substrate or segmented if it is unsupported according to mask layer 19 resulting a plurality of discrete microbeads 21 as shown in FIG. 2F. The mask layer 19 may be removed from the microbead 21, if desired, for example, by wet etching. The etching process can vary depending on polymeric material 17. For example, plasma etching and reactive ion etching (RIE) can be two suitable techniques. The microbeads of the present invention typically can have a diameter of between 1.2 μm and 250 μm. By way of example, a typical sample volume of about 1 μL may contain more than 1,000,000 microbeads of 6 μm each.

If a cross-linkable resin is used, cross-linking can be accomplished by light exposure in the presence of photoinitiators or photo cross-linking agents. In this case, the cross-linkable resin may be used as a “negative resist” in which the material exposed to light can become insoluble to washing solvents. The cross-linking resin can be, but is not limited to, an epoxide-based resist manufactured by Shell® Chemical and others called “SU-8 resist” (see, for example, U.S. Patent # 4,882,245, incorporated herein in its entirety by reference). Other examples of crossed-linkable resins can include silicon-based resins such as silsesquioxanes, silicone polymers such as poly(dimethylsiloxane) (PDMS) and poly(phenylmethylsiloxanes), phenolic polymers (novolac resins), epoxides (such as bisphenol A-based resins), urethane acrylates, and acrylates. Another group of photo cross-linkable materials is based on acrylate, acrylate urethane, or epoxide resins that become crossed-linked with a photoinitiator agent. The constituents of this group are referred to as ultra-violet (UV)-adhesives, examples of which are Norland® optical adhesives. The liquid resin can be a thermoplastic resin such as, but not limited to, polystyrene (PS), poly(methyl methacrylate) (PMMA), polycarbonate (PC), thermoplastic polyimides (Imitec™, Inc. resins), poly(ethylene terephthalate) (PET), polyurethanes (PU), poly(ether ether ketone) (PEEK), and polyethylene (PE). If a photoresist is used, a photomask can be used instead of mask layer 19. It should be noted that, in the illustrative embodiment of the present invention, a negative photoresist could require a photomask or mask layer 19 masking the regions between patterned microbeads 21 instead of masking the areas directly above the patterned microbeads 21 as shown in FIG. 2D. A positive resist can also be used, but in this case the optical masking can be achieved as shown in FIG. 2D since the light-exposed regions dissolve in the developer solution.

Continuing to refer to FIGS. 2A-2E, release/sacrificial layer 15 can be made from a thin fluorinated layer, deposited by conventional fluorinated silane-based monomers, that may not be sacrificed for the release of the microbeads 21. Another example of release/sacrificial layer 15 could be a polymer that is soluble in organic solvents such as xylene, toluene or acetone and that can be deposited by spin casting. Release/sacrificial layer 15 could be formed by passivating the master substrate 11 by the gas phase deposition of a long-chain, fluorinated alkylchlorosilane (CF₃ (CF₂)₆(CH₂) ₂SiCL₃) (see as an example, for illustrative purposes only, Release Layers for Contact and Imprint Lithography, Resnick, Mancini, Sreenivasan, Willson, incorporated herein in its entirety by reference). Also, solution-cast release compounds are available such as, for example, Solvay Solexis Fluorolink®, which can reduce surface energy and impart to the surface the combination of characteristics such as oil/water repellency, easy stain removal, anti-adhesion, and self-lubricity properties. Yet another example of release/sacrificial layer 15 is a positive resist that may not be cross-linked at a later stage and can be soluble in acetone. Since the release/sacrificial layer 15 should be compatible with the processing steps required for imprinting and patterning the microbead material 17, care should be taken to choose the release/sacrificial layer 15 judiciously. For instance a photoresist may not be a suitable release layer for a thermally cross-linked polymeric material since the photoresist may become insoluble after heating above 120° C. For low-temperature UV cross-linked patterning, either of a soluble polymer or light-unexposed positive resist may be suitable for the release/sacrificial layer 15, in addition to a thin fluorinated layer (see, for example, Introduction to Microlithography, Second ed., edited by L. F. Thompson, C. Grant Willson, and M. J. Bowden, ACS Professional Reference Book, American Chemical Society, Washington D.C., 1994, incorporated herein in its entirety by reference).

Referring to FIG. 2D, although a single layer of polymeric material 17 is shown, there may be no restriction on the number of layers used. It may be possible for polymeric material 17 to contain layers (or be coated by layers) that can be made from dielectric (non-conducting) materials other than polymeric materials (materials dispensed in liquid form—spray coating, spin casting dipping, etc), such as SiO₂, TiO₂, tantalum pentoxide, aluminum silicate, and titanium nitride. In the case of these dielectric materials, layering can be accomplished using low-temperature deposition and vacuum methods such as sputtering, plasma enhanced chemical vapor deposition (PECVD) and sol-gel chemistry that are compatible with organic layers. These dielectric materials can have different refractive indices relative to polymeric materials, and can be used to provide a wider range of refractive indices for implementing diffractive optics and direct readout with a microscope. A wider range of refractive indices can enable the possibility of narrow-band “dielectric stack” type mirrors (as opposed to wide-band metallic mirrors). In addition to enabling diffractive optics, dielectric materials can also provide a variety of surfaces, beyond that of polymeric materials, for adsorption and immobilization of ligands and analytes, and thus can offer more diversity for immobilization of ligands and analytes as well as widening the range of conditions in which layers can be used (e.g. Al₂O₃ for pH greater than 9).

Further referring to FIG. 2D, the microbeads may also include a transducing layer that may be polymeric, metallic, or dielectric inorganic material, such as TiO₂, SiO₂, Al₂O₃, tantalum pentoxide, TiN, or aluminium silicates, that is detectable by any chemical or physical means, including electromagnetic, spectroscopic, chemical, photochemical, chemiluminescent or mechanical response. The transducing layer may be of silver, gold, copper, nickel, palladium, platinum, cobalt, rhodium, and iridium. Also useful in the context of the present invention can be metal-organic compounds capable of emitting electromagnetic radiation, such as, for example, aluminum tris (8-hydroxyquinoline) and those described in U.S. Pat. No. 6,303,238 (Thompson et al.), incorporated herein in its entirety by reference. The transducing layer may also be a photoluminescent material such as, for example, 8-hydroxyquinoline aluminium chelate (Alq3), N-p-methodxylphenyl-N-phenyl)-p-methodoxylphenyl-stryrylamine (SA), diphenyl-p-(t-butylphenyl-1,3,4-oxadiazole (PBD), and 4-dicyanomethylene-2-methyl-6-(p-dimenthyaminostyrylk)-4H-pyran (DCM).

Referring now to FIGS. 2G-2L, an alternate embodiment of the encoded patterned microbead is shown in which the microbead 21 can be micropatterned by replication, achieved in this case by embossing. The encoded patterned microbead 21 can be embossed by pressing a polymeric material 17 (using a press) against a master substrate 11 or die containing relief patterns 13, shown in FIG. 2G, and a release layer (not shown) to separate the master substrate 11 from the patterned polymeric material 17. The master substrates 11 in FIGS. 2A and 2G show just one depth level of patterning, but the master substrate 11 may contain two or even more levels of pattern depths. The polymeric material 17 can be a free-standing substance such as a thermoformable polymer (e.g. amorphous PEEK) or it can be a formable film on optional support substrate 12, shown in FIG. 2H, such as a polyimide resin on a Si wafer or a TiO₂ film on a glass, quartz, or Si substrate. Referring to FIG. 21, while the polymeric material 17 is pressed against the master substrate 11, the temperature may be raised to above T_{g} or the soft material undergoes a chemical change (e.g. photo cross-linking) that can raise the T_{g} above the temperature of the embossing press. Referring to FIG. 2J, the impression of the master substrate 11 can be made and the polymeric material 17 can be released from the master substrate 11 with a pattern such as microbead encoding pattern 24 imprinted on polymeric material 17. Referring to FIG. 2K, an etching tool can be used to preferentially etch (remove) portions of the polymeric material 17 to induce optical changes near the surface (or the bulk) of the polymeric material 17. A laser can also be used to permanently mark the surface or bulk of an inorganic film. After creating the pattern, the polymeric material 17 can then be diced into supported microbeads 14 using etching as discussed above or by laser ablation. Implicit in this process may be a sacrificial layer between optional support substrate 12 and polymeric material 17 that can allow removal of supported microbeads 14 from optional support substrate 12, shown in FIG. 2L.

Alternatively, the encoded patterned microbead can be created by lithography that may be based on modifications of a bilayer imprint process known as Step and Flash Lithography (SFIL) (for example, see published United States Patent Application 2001/0040145, and Step and Flash Imprint Lithography: A New Approach to High-Resolution Patterning, Colburn, M. et al., Texas Materials Institute, The University of Texas at Austin, Austin, Tex. 78712, both of which are incorporated herein in their entirety by reference). In the standard approach a transparent master substrate, treated with release layer, can be placed against a substrate (for instance a Si wafer) having an organic polymeric transfer layer on top of it. An etch barrier (liquid to start with), typically a UV polymerizable organosilicon material, can be infused by capillary forces between the master substrate and the polymeric material, then irradiated with UV light through the transparent master (for instance an etched quartz wafer). The master can then be removed leaving a plurality of patterned regions made from the polymerized or crossed-linked organosilicon material, and the transfer layer material can be plasma oxygen etched. This method can be used to produce structures with a high aspect ratio.

Still further alternatively, the encoded patterned microbead can be created by photo bleaching according to a method described in PCT patent application WO 00/63695 and Scanning the Code, Modern Drug Discovery, February 2003, both of which are incorporated herein in their entirety by reference. Photobleaching involves controlled bleaching of the microbeads, which can be formulated of a bleachable substance such as, for example, a material that can bind a fluorescent dye physically or chemically, to form patterns that can be read in various ways such as, for example, raster- and laser-scanning.

These methods can be adapted to the fabrication of microbeads in several ways. One approach might be to fabricate a master having a multi-level depth pattern, for example, a shallow pattern for defining the microbeads on the “transfer layer”, and a deeper pattern defining the code for each microbead. An alternative method could be to use two masters and create the patterns sequentially. The first master can define the perimeter of the microbeads, and the second master can define the microbead encoding. After etching the organic layer with an oxygen-rich plasma through the stop layer, a plurality of separate bilayer encoded regions may remain on the supporting wafer. Here the process can follow one of three paths: (a) the composite bilayer structure made from the transfer layer and the organosilicon encoded layer can be lifted jointly from the substrate by etching the substrate or by dissolving a sacrificial layer between the substrate and the transfer layer; (b) the transfer layer can be dissolved, releasing encoded microbeads made from the organosilicon polymer layer (in this case, additional layers may be added to the microbeads by vacuum deposition techniques before the transfer layer is removed); or (c) the support wafer can be anisotropically etched using RIE, releasing composite organosilicon/organic/wafer-material microbeads.

Referring now to FIGS. 2M-2S, an intermediate embodiment, similar to SFIL, between casting the polymeric material 17 on a patterned master substrate 11 (FIGS. 2A-2F) and embossing polymeric material 17 (FIG. 2G-2L) can be achieved by coating a support substrate 12 with a soft moldable polymeric material 23 and then imprinting a two-level depth patterned master substrate 11, shown in FIG. 2M, against the composite of the support substrate 12 and the soft moldable polymeric material 23, shown in FIG. 2N. Referring to FIG. 2M, the two-level pattern can include a first shallow pattern 27 that forms the perimeter of the microbeads 21 and a second deep relief pattern 28 that forms the encoding of the microbead 21. Two levels are shown herein for illustrative purposes only, the invention is not limited to two levels of depth. Shown in FIG. 2P, the polymeric material 17 can become cross-linked by heat or light and the imprint can become permanent. The master substrate 11 can be removed, shown in FIG. 2Q, leaving a plurality of supported microbeads 14 on the support substrate 12, shown in FIG. 2R. Finally, in FIG. 2S, microbeads 21 may be freed from the support substrate 12 by use of a release layer (not shown).

Referring now to FIGS. 2T-2W, laser ablation can be used to create the pattern for the microbeads. In this process, referring to FIG. 2T, polymeric material 17 can be deposited onto a support substrate 12, for example, polyimide on Si. Referring to FIG. 2U, a laser may be used to inscribe the polymeric material 17 with encoding pattern microbead 24, and possibly may also be used to cut out regions on the polymeric material 17 corresponding to the individual microbeads 21, shown in FIG. 2V. In case a free-standing polymeric material 17 is used, the laser may be used to cut out and free the individual microbeads 21, shown in FIG. 2W. Implicit in this process, a sacrificial layer can be placed between support substrate 12 and polymeric material 17 as above.

In a variation on the method of FIG. 2T-2W, laser writing can be used to create the microbead encoding pattern 24. In this case, a thin film of a substance such as TiO₂ may be deposited by sputtering or by a sol-gel process onto support substrate 12 such as, for example, glass, polymer, or Si. The film may be further patterned into a plurality of regions corresponding to the microbeads. A UV laser may be used to permanently inscribe the polymeric material 17. The supported microbeads 14 may then be defined by a method such as, for example, dry etching. Afterwards, the microbeads 21 may be freed from the support substrate 12 by use of a release/sacrificial layer (not shown). In general, in all the processes described with respect to FIGS. 2A-2W, before the microbeads 21 are released, additional layers could be added, including layers of metals and dielectric materials, depending upon the application.

Referring now to FIG. 2X, a protective layer 53, which can optionally be laid on top of a transducing (e.g. reflective) layer 55, is shown. It is also possible that digital data layer 57, alone, can act as a transducing layer. Alternatively, digital data layer 57 may contain photo-sensitive dyes that can be burned or photobleached with a laser. A transducing system may be formed when digital data layer 57, physically marked in a desired pattern to reveal (or block) a reflective, photoluminescent or absorbing pattern, either may cooperate with transducing layer 55 or may act as a transducing layer itself. Preferably, the transducing system, possibly including transducing layer 55 and/or digital data layer 57 can produce a detectable response signal when exposed to energy. Preferably, the detectable signal produced by the transducing system can be read by an optical reader as binary data. Suitable materials for transducing layer 55 can include films containing silver, indium, antimony, and tellurium. Alternatively, digital data layer 57 may be coated with photo-sensitive dye that may be burned with a laser according to the desired pattern of 1's and 0's. Darker and lighter areas, when read, may be understood as binary data. Still further alternatively, phase change technology, involving laser-heating the alloy to two different temperatures, can produce two different crystalline structures. A third laser temperature can be used to read the binary data from the alloy. Using this technology, data may be written more than once, in fact up to 1000 times. Data may be stored more densely by several conventional methods. For example, data may be stored more densely using well-known methods such as Fluorescent Multilayer Optical Data Storage devices (see for example, but not limited to, published United States Patent Application 2002/0098446, and U.S. Pat. No. 6,338,935, both of which are incorporated herein in their entirety by reference). Referring now to FIG. 2Y, as described previously, microbeads may be etched from the larger substrate of polymeric material 17, and may be released as individual microbeads 21, shown in FIG. 2Z.

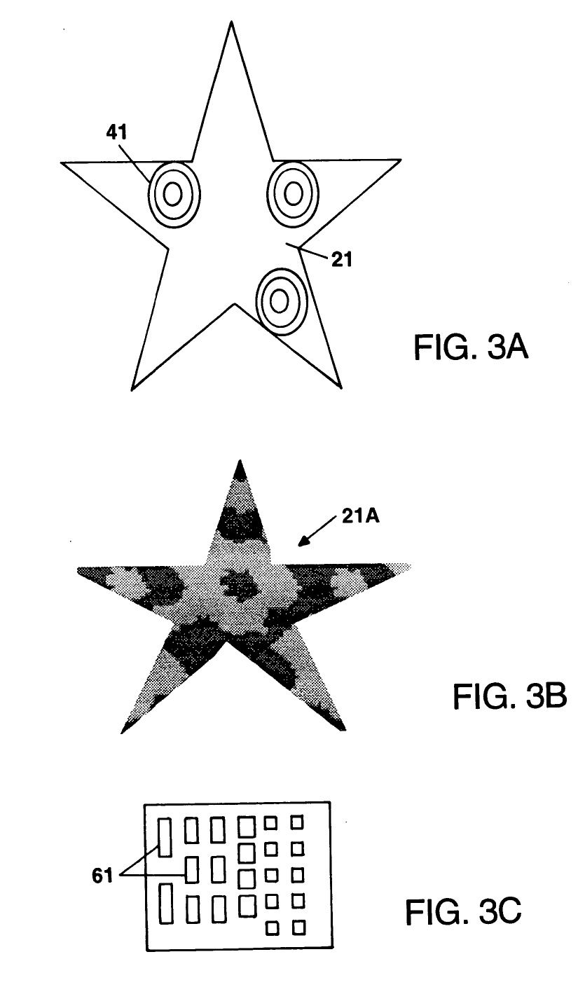

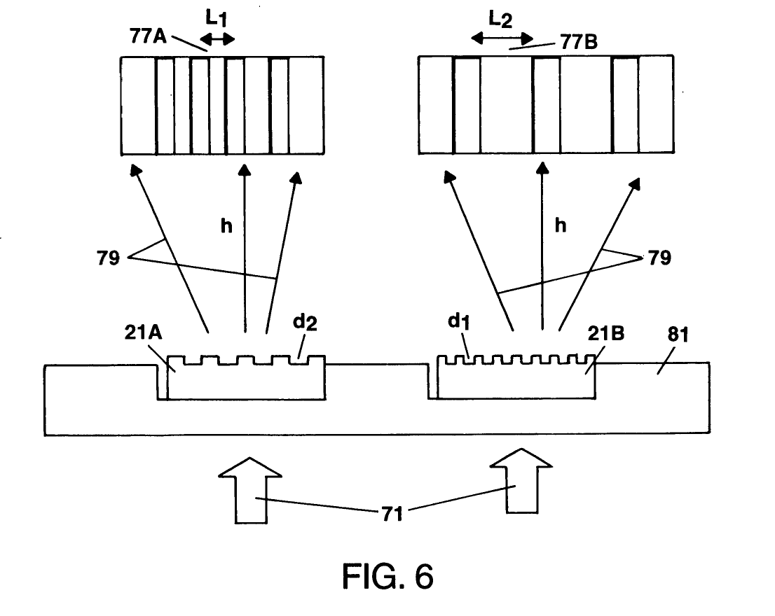

Referring now to FIG. 3A, shown is an encoded microbead 21 created through techniques shown in FIGS. 2A-2W. In FIG. 3A, the circular optical grating 41 corresponds to a type of microbead encoding pattern 24 (FIG. 2D) in which the circles represent ridges and troughs corresponding to desired patterns of constructive and destructive interference. In circular optical grating 41, the difference between up (e.g. light) and down (e.g. dark) regions, is given by de=(λ/2)/(n−n₀), where n is the refractive index of the polymeric material 17 and n₀ is the refractive index of a medium through which the depth of the pattern is measured. For example, when a polymeric material 17 has a refractive index of 1.4, and the medium is air (n₀=1), if green light (λ=550 nm) is used, then the depth of the pattern, de, may be ˜0.7 μm. If the medium is water (n₀=1.33), de ˜3.9 μm. On the other hand, if there is a layer TiO₂ (n ˜2.8) on top of polymeric material 17, and the medium is air, de ˜0.15 μm. If the medium is water, de˜0.18 μm. The circularly invariant diffractive optics pattern is shown in which various ring spacings d (or pitch, see FIG. 6, d₁ and d₂) in circular optical grating 41 may be used to create and later interpret the resulting pattern obtained by the reading method later described. Other methods can be used to inscribe the microbead encoding pattern 24 such as photolithography, differential etching methods, or holographic patterning beams acting on a photochromic or temperature/optically sensitive material dispersed in the polymeric material 17 or as part of the structure of polymeric material 17. Using any of these methods, it may be possible to write optically contrasting regions in three dimensions in the bulk of every microbead. The concentric circular pattern of FIG. 3A, however, is only an example of a possible pattern that can be read using the reading process of the illustrative embodiment of the present invention (later described). Furthermore, in general, the encoding of the microbeads can take the form of varying the sizes and/or shapes of the microbeads. For example, microbeads can take circular shapes of size 10, 20, or 30 μm, squares shapes of size 10, 20, and 30 μm, star-shapes with four points, star-shapes with five points, etc. These examples are given for illustrative purposes only and are not intended to limit the size or shape of the encoded microbeads of the present invention.

Referring now to FIG. 3B, a portion of a repeating pattern of light spots is shown on microbead 21A. This complete pattern corresponds to a “unit cell” and may be repeated periodically over at least part of the layer of polymeric material 17. The lateral dimensions of the “unit cell” can determine the pitch of light diffraction that in turn determines the distance between features of the diffracted array at a given distance from the microbead (this distance corresponds to L₁ and L₂ diameters of the pattern in FIG. 6). Any portion of the pattern that is illuminated may create the array of light spots, and thus the beam cross-section can be made smaller than the microbead area without affecting the shape of the array of light. The array of light spots is detected, in the illustrative embodiment, with a 2-d charge-coupled device (CCD), to which data may be applied well-known algorithms to produce the resulting microbead identification. The microbeads could be patterned identically but the spacing of the pattern could internally vary such that a wider or narrower distance between the beams of light (from the array) could be generated by the microbead.

FIG. 3C shows an exploded view from the surface layer 59 (FIG. 2Y) of a microbead prepared according to FIGS. 2X-2Z in which pits 61 (see also FIG. 2Y) are clearly shown. In general, the transducing layer 55 (FIG. 2Y) can be any suitable material that is detectable by any chemical or physical means, including electromagnetic, spectroscopic, chemical, photochemical or mechanical response. Preferably, the transducing layer 55 (or the digital data layer 57) produces a detectable response signal to exposure to energy. A detectable response signal, used herein, is meant to include any emission of energy, including elastic or inelastic electromagnetic radiation (visible or infrared or ultraviolet light)- and any other signal or change in signal emanating from the transducing layer 55 (including diffraction) and/or absorption in response to exposure of the transducing layer 55 to energy. Preferably, the detectable signal produced by the transducing layer 55 is an electromagnetic emission or absorption. Suitable transducing layer 55 materials can include films containing silver, gold, copper, nickel, palladium, platinum, cobalt, rhodium, and iridium, as well as dielectric layered materials such as TiO₂, SiO₂, Al₂O₃, tantalum pentoxide, TiN, and aluminium silicates. Also useful in the context of the present invention are metal-organic compounds capable of emitting electromagnetic radiation, such as, for example, aluminum tris (8-hydroxyquinoline) and those described in U.S. Pat. No. 6,303,238, incorporated herein in its entirety by reference.

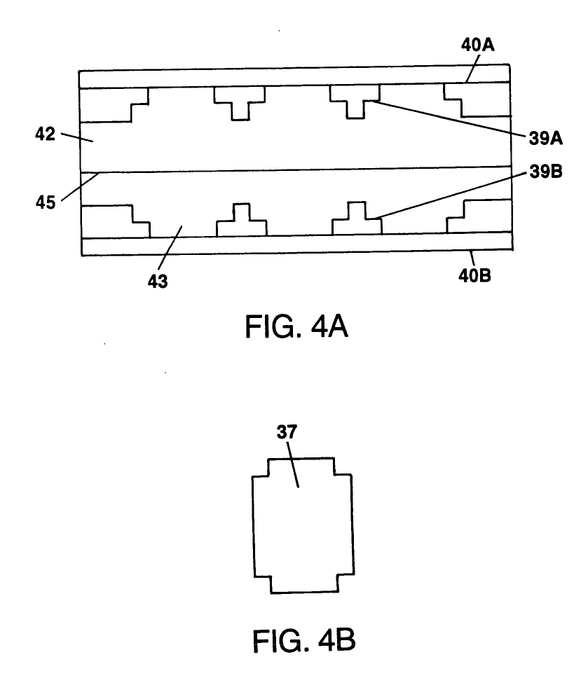

Referring now to FIG. 4A, a first embossed polymeric material 42 and a second embossed polymeric material 43 may be brought together at interface 45 and bonded so that their surfaces 39A and 39B to be patterned are towards the outside of the bond. A single film can be double-embossed by laying two masters 40A and 40B, instead of a single master, against a flat platen at opposite sides of the film, surfaces 39A and 39B, during the pressure-temperature cycle. Referring now to FIG. 4B, after dicing the film, the “two-sided” microbeads 37 have patterns on both faces. Implicit in this process are release layers on patterns 39A and 39B as described above.

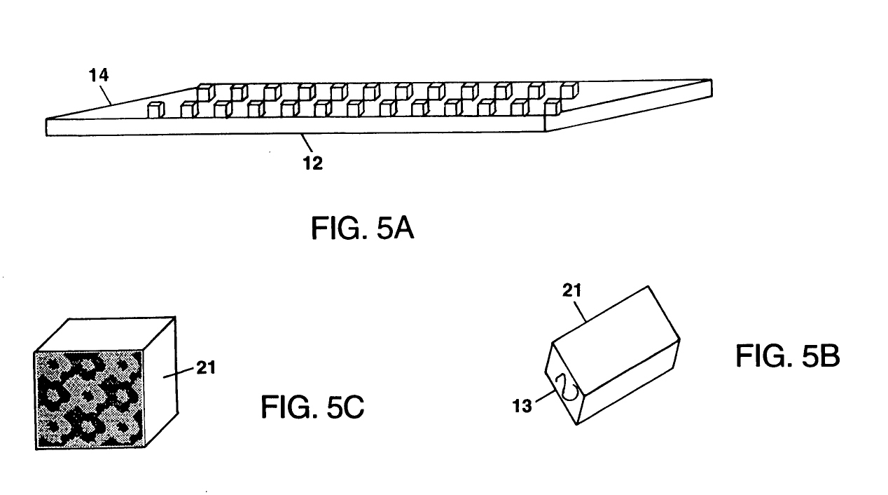

Referring now to FIG. 5A, supported microbeads 14 are in position for release from the support substrate 12. Microbead 21, shown in FIG. 5B, is encoded (in this case with a simple letter “S”), but it should be clear that a virtually unlimited supply of microbeads 21 could be specially encoded with a virtually unlimited number of unique encoding microbead patterns 24. FIG. 5C illustrates the pattern described in FIGS. 2X-2Z. Additionally, microbead 21 can be marked, after binding with an analyte (or target molecules) and identified by the emission of dyes or luminescent molecules associated with the analytes, with an optical or magnetic characteristic that could simplify or assist the process of isolation of the given microbead for further analysis or product purification.

Using an automated microscope, for example, near-field reading of the encoding of the present invention may be accomplished by using shapes, such as triangles, circles, squares, crosses, diamonds, parallelograms, semicircles, etc., to distinguish the microbeads one from another. Also, shapes could be used in combination with color dyes, color absorbing dyes (or pigments), or dielectric coatings to create an interferometric or holographic color pattern. Conventional pattern-recognition techniques can then be used to read the encoding, and multiplexing by both shape and color can be accomplished. Another method for reading could include the use of a confocal microscope in which microbeads could be spread on a substrate and read. Likewise, if a fluorescent microscope is used, only microbeads with fluorescence on them might be chosen. From the microbeads that are chosen in these ways, automatic or manual pattern recognition can be used to read the pattern on the microbeads.

Yet another method of reading could include a combination of microbead construction and a near-field optical device or far-field optical array sensor. In this method, metallic layers or dielectric stacks may be used in microbead construction, and monochromatic or multicolor light and filters may be used in a microbead reader such that the pattern on the embossed microbeads may be read either by a near-field optical device, or with a far-field optical array sensor. The cross section of the illuminating beam should be comparable in size to the microbead so as to illuminate and identify one microbead at a time. Alternatively an array of beams (each with cross section comparable to the size of the microbead) may be used to simultaneously identify a plurality of beads, each microbead being imaged independently from each other.

Yet another method for reading involves illuminating an entire substrate covered with microbeads at once so that every pattern is seen. If a dichroic filter is added between the substrate and the sensor, the elastic diffracted light (i.e. with the same spectral characteristics as the incident light) can be blocked, allowing only the light emitted by dyes or luminescent molecules associated with the analyte molecules bound to the microbeads to reach the detectors. The diffractive patterns from microbeads that do not bind analyte molecules can thus be blocked by the filter. Further, with several thousand microbeads on a substrate, even if the luminescence of dyes or luminescent molecules associated with analytes from a single microbead might be faint, the illumination that results may be the sum of the illuminations of each microbead, thus making far-field reading a possibility.

Referring now to FIG. 6, a beam of light 71 is projected at an angle onto microbead 21A and 21B which may be etched, molded, embossed, etc. with variously-spaced gratings. The diffracted light from the beam 71 can form an image on a detector arrays 77A and 77B (such as a 1-d or 2-d CCD detector array) where the image may be recorded in the conventional way. In operation, the spacings d₁ and d₂ may work cooperatively under beam 71 to form a diffracted light image that intersects the CCD detector arrays 77A and 77B located at a distance h above the substrate, making lines of light of spacings L₁ and L₂ on the plane of the CCD detector arrays 77A and 77B. As shown here, for example, if the CCD detector arrays 77A and 77B are one-dimensional (linear) arrays, the projected light may intersect at two or more points along the array separated by the distances L₁ and L₂. These variables are related by the Bragg diffraction condition L_{1/2}˜λ₀h/d_{1/2}. The distance h can be small, for example, several hundred microns, or quite large, several millimeters. A series of lines or spots of light from each microbead could be created by patterning the microbead appropriately. In a single-microbead reading configuration, the emission from dyes or luminescent molecules associated with analytes bound to the microbead can be read through a dichroic filter using a conventional fluorescence imaging system (not shown), and simultaneously the size and spacing of the lines or spots can be read at either the same wavelength of the dye emission or at any another wavelength.

Continuing to refer to FIG. 6, in a different arrangement, multiple microbeads could be illuminated and imaged simultaneously. Here the CCD detector arrays 77A and 77B can be located at several millimeters away from substrate 81 to allow for integration of the emission of multiple microbeads. The readout may be made through a dichroic filter (not shown) that isolates the emission from dyes associated with the analytes bound to the microbeads 21A and 21B. In this case the image consists of multiple bands or spots spaced with different pitch (distances L₁ and L₂ between lines) (each corresponding to a class of microbeads with the same pattern and the same ligand), and the corresponding intensities may be determined by the amount of analyte bound to each class of microbead. When a single class of microbeads binds to the analyte, there is a single pattern, corresponding to the class of microbead that successfully captured the analyte. In the case of having several microbeads capturing some of the analyte molecules, multiple patterns of lines or spots may be seen. None of the non-binding microbeads should be imaged since the dichroic filter rejects the light arising from elastically diffracted light (i.e. with the same spectral characteristics as the incident light).

Referring further to FIG. 6, microbeads can be encoded such that they can be read by reflection or transmission (through the substrate), i.e. microbeads can be illuminated from the bottom or from the top and reading can be accomplished through the substrate. When reading by reflection, one or both sides of the microbeads are encoded with the same code, light is introduced from the top, and impinges upon the microbeads at an angle. The incident light could be diffracted from the beads in reflection mode. Note that for simplicity FIG. 6 shows a 1-d grating, but the concept can be expanded to any number of dimensions without changing the fundamental aspects of the invention.

Although the invention has been described with respect to various embodiments, it should be realized that this invention is also capable of a wide variety of further and other embodiments within the spirit and scope of the appended claims.

What is claimed is:

1. A microbead particle system for bioassay comprising:
at least one microbead particle made of polymeric material;
a pattern encoded on at least one portion of said at least one microbead particle;
a selected geometry effectively associated with said at least one microbead particle, said geometry capable, alone or with other artifacts, of identifying said at least one microbead particle; and
means effectively associated with said at least one microbead particle for enabling or enhancing chemical conjugation between said at least one microbead particle and a ligand.

2. The microbead particle system as defined in claim 1 wherein said polymeric material is selected from the group consisting of thermoplastics, thermosets, photocrosslinkable resins, photopolymerizable resins, and organosilicon resins.

3. The microbead particle system as defined in claim 1 wherein said pattern is encoded in at least one dimension or within said portion.

4. The microbead particle system as defined in claim 1 further comprising at least one layer of material on or within said polymeric material, said at least one layer of material including material selected from the group consisting of dielectric materials, SiO₂, TiO₂, tantalum pentoxide, aluminum silicate, titanium nitride, metals, silver, gold, copper, nickel, palladium, platinum, cobalt, rhodium, iridium, photoluminescent compounds, aluminum tris (8-hydroxyquinoline), hydroxyquinoline aluminium chelate, N-p-methodxylphenyl-N-phenyl-p-methodoxylphenyl-stryrylamine, diphenyl-p-t-butylphenyl-1,3,4-oxadiazole, 4-dicyanomethylene-2-methyl-6-(p-dimethylamino styryl)-4H-pyran, and polymer blends containing photoluminescent polymers, poly(phenylenevinylenes), poly(fluorenes), and polythiophenes.

5. The microbead particle system as defined in claim 4 wherein said at least one layer of material is electromagnetically transducing, said at least one layer of material having a measurable response to electromagnetic excitation, said measurable response formed according to said pattern.

6. The microbead particle system as defined in claim 4 wherein said at least one layer of material includes at least one surface suitable for chemical conjugation with a ligand.

7. The microbead particle system as defined in claim 1 wherein said pattern is symmetrical.

8. The microbead particle system as defined in claim 1 wherein said pattern is a preselected pattern capable of generating a diffractive image.

9. The microbead particle system as defined in claim 1 wherein said pattern comprises at least one unit cell, said at least one unit cell being repeated on at least part of said at least one portion, said pattern capable of generating a diffractive image.

10. The microbead particle system as defined in claim 9 wherein said pattern is capable of generating the diffractive image as long as a region of said pattern is illuminated by a beam having at least the same size as said at least one unit cell, said at least one unit cell capable of being illuminated at an angle.

11. The microbead particle system as defined in claim 1 wherein said pattern comprises a plurality of regions, said plurality of regions being capable of producing a plurality of electromagnetic responses, said plurality of electromagnetic responses generating a binary code.

12. The microbead particle system as defined in claim 11 where said plurality of electromagnetic responses is selected from the group consisting of reflectivity, light absorption and photoluminescence.

13. The microbead particle system as defined in claim 1 wherein said geometry comprises a pre-selected surface shape and size, said geometry enabling seating in a receiving substrate in a manner effective for particle identification.

14. The microbead particle system as defined in claim 13 wherein said pre-selected surface shape and size is selected from the group consisting of triangles, circles, squares, crosses, diamonds, parallelograms, and semicircles, wherein said pre-selected surface shape is used in combination with a treatment selected from the group consisting of color dyes, color absorbing dyes, pigments, and dielectric coatings, said treatment creating an interferometric or holographic color pattern.

15. The microbead particle system as defined in claim 1 wherein said at least one portion is a transducing layer or a digital data layer, said transducing layer or digital data layer further comprising:
a protective layer laid on top of said transducing layer or said digital data layer;
wherein said digital data layer, either cooperating with said transducing layer or acting as said transducing layer, produces a detectable response signal when exposed to energy, wherein said transducing layer or said digital data layer is made of material selected from the group consisting of silver, indium, antimony, and tellurium, wherein said transducing layer or said digital data layer is coated with photo-sensitive dye that is burned with a laser according to a pre-selected pattern of 1 's and 0's.

16. The microbead particle system as defined in claim 1 wherein said pattern represents ridges and troughs corresponding to pre-selected constructive and destructive interference patterns, a relationship between said ridges and troughs being a function of refractive index of said polymeric material, refractive index of a medium through which the depth of said pattern is measured, and the wavelength of light impinging on said pattern.

17. The microbead particle system as defined in claim 1 wherein said at least one portion further comprises:
a first embossed polymeric material having a first inner surface opposing a first patterned surface; and
a second embossed polymeric material having a second inner surface opposing a second patterned surface,
wherein said first inner surface forms a bond with said second inner surface.

18. The microbead particle system as defined in claim 1 further comprising said at least one microbead particle being marked after binding with an analyte, said at least one microbead particle being identified by the emission of dyes or luminescent molecules associated with the analyte.

19. The microbead particle system as defined in claim 1 wherein said at least one portion comprises a metallic layer or a dielectric stack

20. A method for fabricating at least one polymeric microbead comprising the steps of:
creating a patterned master substrate having at least one pattern and at least one shape, the at least one pattern having at least one level of pattern depth, the at least one shape enabling identification and proper seating in a receiving substrate;
applying polymeric material to the patterned master substrate to form at least one patterned polymeric microbead or at least one patterned microbead precursor;
partitioning the polymeric material to form the at least one polymeric microbead; and
releasing the polymeric material from the master substrate.

21. The method as defined in claim 20 wherein said step of applying polymeric material to the patterned master substrate is performed according to a process selected from the group consisting of embossing, casting a liquid resin onto the patterned master substrate, injection molding a liquid resin onto the patterned master substrate, and infusing a liquid resin into a gap formed between the patterned master substrate and a second substrate.

22. The method as defined in claim 20 wherein said step of partitioning the polymeric material to form the at least one patterned polymeric microbead is a process selected from the group consisting of dry etching the polymeric material, cutting the polymeric material using laser ablation, and dissolving the polymeric material surrounding the at least one patterned polymeric microbead.

23. The method as defined in claim 20 wherein said step of creating at least one level of pattern depth comprises:
creating a first depth that defines a plurality of features; and
creating a second depth that defines at least one labeling code, the second depth being deeper than the first depth.

24. The method as defined in claim 20 wherein said step of applying the polymeric material to the patterned master substrate further comprises the steps of:
casting a liquid resin onto the patterned master substrate; and
hardening the liquid resin to form a micropatterned polymeric substrate.

25. The method as defined in claim 20 wherein said step of applying the polymeric material to the patterned master substrate further comprises the steps of:
injection molding a liquid resin onto the patterned master substrate; and
hardening the liquid resin to form a micropatterned polymeric substrate.

26. The method as defined in claim 25 further comprising selecting the liquid resin from the group consisting of epoxide-based resist, silicon-based resins, silsesquioxanes, poly(dimethylsiloxane) (PDMS), poly(phenylmethylsiloxane), phenolic resins, novolac resins, epoxides, bisphenol A-based resins, urethane acrylates, acrylates, ultra-violet adhesives, optical adhesives, thermoplastic resins, polystyrene, poly(methyl methacrylate), polycarbonate, thermoplastic polyimides, poly(ethylene terephthalate), polyurethanes, poly(ether ether ketone), and polyethylene.

27. The method as defined in claim 20 further comprising the step of providing at least one layer of material on top of the polymeric material.

28. The method as defined in claim 20 further comprising selecting a material for the patterned master substrate from the group consisting of silicon, quartz, aluminium oxide, glass, metals such as stainless steel, copper, chromium, nickel, and brass.

29. A microbead being formed according to the method of claim 20.

30. A reader for identifying at least one microbead comprising:
a receiving substrate, said receiving substrate including at least one receptor having at least one geometric shape, said at least one receptor capable of receiving at least one microbead with a portion having a geometry corresponding to said substrate receptor geometry;
a magnifier capable of enlarging an optical, electrical, pressure, sonic or magnetic image of the received at least one microbead or a portion thereof; and
a recorder capable of storing an enlarged image of the received at least one microbead or portion.

31. The reader as defined in claim 30 wherein said at least one receptor is selected from the group consisting of a well, a treated portion of said receiving substrate, and a protrusion.

32. A method for identifying at least one microbead comprising:
initially etching a receiving substrate through a first patterned mask, said step of initially etching forming a shaped opening, the shaped opening having a pre-selected geometry;
subsequently etching the receiving substrate through a second patterned mask, said step of subsequently etching enlarging the shaped opening;
creating a master substrate having at least one pattern, the master substrate having the pre-selected geometry, the at least one pattern having at least one level of pattern depth;
applying polymeric material to the master substrate to form the at least one microbead;
partitioning the polymeric material to release the at least one microbead;
releasing the polymeric material from the master substrate;
providing the at least one microbead to the shaped opening; and
viewing the at least one microbead to read the at least one pattern.

33. The method as defined in claim 32 further comprising the steps of:
forming the shaped opening having a top surface and a bottom surface;
forming a beveled edge at the top surface; and
forming the bottom surface smaller than the top surface.
